(19) United States
(12) Patent Application Publication
(10) Pub. No.: US 2015/0038547 A1
(43) Pub. Date: Feb. 5, 2015

(54) Biological Basis of Adult Wellness

(71) Applicant: Rory Powell Blake, Charlotte, NC (US)

(72) Inventor: Rory Powell Blake, Charlotte, NC (US)

(21) Appl. No.: 13/956,327

(22) Filed: Jul. 31, 2013

(51) Int. Cl. A61K 31/401; A23L 2/66; A23L 1/305

(52) U.S. Cl. CPC A61K 31/401; A23L 1/3051; A23L 2/66; A23V 2002/00
USPC 514/423; 548/535; 426/656; 426/590

(57) ABSTRACT

This disclosure is of a lowered absorption of nutrients within the adult digestive tract, more specifically, a decreased mechanical absorption of certain specific amino acids over time; said decreased nutritional uptake decreasing wellness of the individual by the mechanism of depletion of bodily reserves; said lowered uptake being mitigated by a patently unique method to intercede in the nutrient absorption process.

### CROSS-REFERENCE TO RELATED APPLICATIONS

The present application claims priority of U.S. Provisional application 61/741,990 dated Aug. 1, 2012 which is hereby incorporated by reference herein.

Proline Suppresses Apoptosis

WO 2006130190 A2 Jan. 25, 2007 Martin B. Dickman

The discovery that proline is a potent antioxidant and inhibitor of programmed cell death is described,

Improved Amino Acid Solutions for Treatment of Peritoneal Dialysis

WO 1994014430A1 Oct. 23, 2002 Michael R. Jones

The present invention provides a dialysis solution that contains amino acids for treating and/or preventing malnutrition in a peritoneal dialysis patient.

Peritoneal Dialysis Solutions Containing Maltodextrins and amino Acids

WO 1995019778 A1 Jul. 27, 1995 Ron Burke

A peritoneal dialysis solution comprising as osmotic agents approximately 2.0 to about 6.0% (w/v) maltodextrins and approximately 0.25 to about 2.0% (w/v)amino acids.

Zingiber Plant Extract

WO2007056811 A1 May 24, 2007 Clifford J. Hawkins

The present invention relates generally to plant extracts and/or components isolated there from which exhibit desirable properties in relation to therapy.

Prevention of Cellular Senescence in Mammals by Natural Peptide Complexes

U.S. Pat. No. 8,258,343 B1 Sep. 4, 2012 Shyam K. Gupta

Preventing skin aging by targeting multiple causes by a single bullet is of primal scientific and consumer interest.

Histidine Proline Rich Glycoprotein (hprg) as an Anti-Angiogenic and Anti-Tumor Agent

EP 1365804 A4 September 8, 2004 Fernando Donate

Histidine Proline Rich Glycoprotein (HPRG) polypeptides or fragments thereof including pentapeptide fragments and multimers thereof, and other biologically active derivatives of HPRG are anti-angiogenic.

Composition and Methods for Decreasing Muscle Breakdown

WO 1994002139 A1 Feb. 3, 1994 Douglas W. Wilmore

The present invention provides compositions and methods for decreasing tissue breakdown in mammals.

Proline Derivatives and Use thereof as Drugs

EP 1930319 B1 May 2, 2012 Hiroshi Kitajima

The present invention relates to a proline derivative showing a dipeptidyl peptidase IV (DPP-IV) inhibitory activity, which is useful for the treatment or prophylaxis of diabetes, obesity, HIV infection, cancer metastasis, dermopathy, prostatic hyperplasia, periodontitis, autoimmune disease and the like, and a salt thereof.

Proline Derivatives having Affinity for the Calcium Channel Alpha-2-Delta Subunit

WO 2004039367 A1 May 13 2004 David James Rawson

The compounds of formula (I) or a pharmaceutically acceptable salt, solvate or pro-drug thereof, are proline derivatives useful in the treatment of epilepsy,

Systems and Methods Using Nuclear Magnetic Resonance (NMR) Spectroscopy to Evaluate Pain and Degenerative Properties of Tissue

U.S. Pat. No. 8,344,728 B2 Jan. 1, 2013 Sharmila Maiumda NMR spectroscopy is performed on intervertebral disc tissue. Extent of degeneration is based on the NMR

Resveratrol-Containing Compositions and Methods of Use

EP 2584897 A1 May 1, 2013 William F Sardi

A resveratrol-containing composition capable of providing a therapeutic benefit to a subject such as modulation of a biological activity, improving cell transplantation therapy, or improving macular degeneration or dystrophy treatments.

Aprepitant L-Proline Solvates—Compositions and Cocrystals

WO 2013076659 A1 May 30, 2013 Joanne Holland

Aprepitant L-proline solvate compositions and aprepitant L-proline solvate cocrystals are disclosed as well as pharmaceutical compositions containing them. The compositions and cocrystals of the invention are: 1:1:1 aprepitant L-p

Multicomponent Crystalline System of Ezetimibe and Proline

WO 2013014604 A1 Jul. 24, 2012 Andreas Hafner

Provided is a crystalline composition comprising a mixture of a compound of formula 1 (Ezetimibe) and proline or proline derivatives, or a hydrate/solvate thereof, as well as a process for obtaining the same.

Composition of Estrogen and Other Hormones with Ascorbate, Lysine, Proline and Other Substances

WO 2003051371 A1 Jun. 26, 2003 Vadun Ivanov Matthias Rath

Abstract: This invention concerns compositions of estrogen and related hormones in combination with ascorbate, lysine, proline and other substances effecting connective tissues, their ability and their use in contraception,

Substantially Stereomerically Pure Fused Bicyclic Proline Compounds and Processes for Preparing Boceprevir

US 20120289709 Nov. 15, 2012 James LaLonde

The present disclosure provides substantially enantiomerically pure heterobicyclic compounds

Cocrystal of C-Glycoside Derivative and L-Proline

WO 2007114475 A1 Oct. 11, 2007 Masakazu Imamura

A cocrystal of (1S)-1,5-anhydro-1-[3-(1-benzothien-2-ylmethyl)-4-fluorophenyl]-D-glucitol and L-proline. This cocrystal ensures a constant quality, excelling in storage stability, and does not exhibit any moisture absorptivity, being a cocrystal of known compound (A) being suitable as a crystal of raw drug to be finished into pharmaceutical products.

Proline Substituted Cyclosporin Analogues

U.S. Pat. No. 8,349,312 B2 Jan. 8, 2012 Guoqiang Wang

The present invention provides novel proline substituted cyclosporinanalogue compounds, pharmaceutical compositions comprising these compounds and methods of using these compounds for the treatment of disorders and diseases, including immune disorders, inflammatory disorders and viral infections.

Sweetening Agent Containing Glycine, L-Proline and/or L-Hydroxyproline

WO 1995027408 A1 Oct. 19, 1995 Jia Zengshen

There is disclosed a sweetening agent comprising glycine, L-proline, and/or L-hydroxyproline. There is also disclosed a sweetener composition comprising a sweetening agent, glycine as a bulking agent and at least one of L-proline and L-hydroxyproline.

Composition Comprising L-Proline for the Treatment of Vitiligo

EP 1661565 B1 Oct. 24, 2007 Barca Ileana

The invention relates to the field of dermatology, more particularly, to a composition that can be used in the area of cosmetology and medicine and which is useful for the treatment of lesions caused by vitiligo.

Mannitol and/or Proline for Prevention and Treatment of Ageing Related Symptoms

WO 2007133076 A2 Nov. 22, 2007 Jan Hendrik Jozef Hoeijmakers

The current invention provides new methods and means for the prevention and treatment of ageing-related symptoms and diseases.

Mannitol and/or Proline for Prevention and Treatment of Ageing Related Symptoms

US 20090247476 A1 May 14, 2007 Ingrid van der Pluijm

The current invention provides new methods and means for the prevention and treatment of ageing-related symptoms and diseases.

Use of mannitol in anti ageing treatments and Selection of anti ageing compounds using non human animal models with mutations in the DNA repair mechanisms.

EP 2218449 A3 May 18, 2011 Jaan 011e Andressoo

The current invention pertains to a method for screening and discovery of compounds capable of inhibiting, preventing, delaying or reducing genome maintenance disorders and consequences thereof, in particular ageing related symptoms and disorders.

Prematurely Ageing Mouse Models for the Role of dna Damage in Ageing and Intervention in Ageing-Related Pathology.

US 20090077676 A1 Nov. 15, 2005 Jan Hendrik

The current invention pertains to a method for screening and discovery of compounds capable of inhibiting, preventing, delaying or reducing genome maintenance disorders and consequences thereof, in particular ageing related symptoms and disorders.

### BACKGROUND OF THE INVENTION

Copyright notice 2013 Rory Blake. All rights reserved. Laws of nature cannot be patented. A portion of the disclosure of this patent application and patent document contains material that is subject to copyright protection. The copyright owner has no objection to the non-commercial facsimile reproduction of the patent document or of the patent disclosure as it appears in the United States Patent and Trademark Office records, database or files.

The field of the invention is nutrition. The express field of the invention is in the improvement in health of the individual by correction of nutritional uptake imbalance.

In nature, the longest-lived mammals are of the larger species. Genetically, they make a larger investment in gestation. The genetic payback is a larger and more sophisticated animal. With an increase in size comes a need for an increased capacity to absorb nutrients. Larger animals intrinsically have a much greater need for nutrients, both to supply energy -and for the maintenance of their much greater bulk. Without both the availability of additional nutrition and an additional capacity to absorb this additional nutrition the animal would go into a steady decline.

As it is programmed into their genes, larger animals maintain an intrinsic capacity to uptake more nutrients than do smaller species. They can also retain larger reserves of critical nutrients. Just as printer cartridges may be quickly emptied with heavy use or more slowly depleted with lighter use, this genetic capacity for greater intake can be reconfigured. It may proceed normally, or if need be, configured into a lower daily intake over a longer period of time.

In addition to size, the longest living mammals in nature have in common being at the top of the food chain. The longest living animals share a characteristic of not being constantly under physically and emotionally stress. Synthesis of signaling peptides, as is required to produce neural hormones, takes away from critical amino acid reserves. All animal groups exhibit individuals within their species attaining a great age. Within any species, environmental modifications to ease the individual's circumstances can extend that individual's lifespan due to prolongation of critical amino acid reserves.

The success of this strategy is correlated with the species genetic potential for physical size. The potential for larger size carries with it the potential to ingest and to absorb more nutrients. This becomes an increased potential to be able to continue forward. The animal who can continue forward with somewhat lowered quantities of nutrients will then continue forward over a much longer period of time.

In England, a goldfish, confined to a small bowl was reported to have lived for over 40 years. A goldfish characteristic is that they do not grow to their maximum size when confined to a small environment. Individuals, such as this goldfish, however, always retain some amount of this unused genetic capacity. This unused nutritional uptake capacity is what then becomes the extra capacity to continue forward.

In humans, the smaller female tends to live a few years longer than the typically much larger, or much more physical male of the species. In general, a female eats somewhat less than a male. This is especially true if a male is employed in a highly physical occupation, such as that of manual labor. Heavy, manual labor requires the uptake and expenditure of more energy. Given the same generic capacity for the intake and absorption of nutrients, the female would be able to intake smaller meals over a much longer period of time before reaching the species-specific intake limit. Sharing a smaller size within a larger species would seem to aid in both health and wellness. A smaller body mass with the retention of the larger species-specific capacity for absorbing nutrition is responsible for the above life-extending phenomenon.

In France, long leisurely meals and the occasional bon-bon for a snack are highly prized traditions. Spreading nutritional intake equally over the day has long been thought to be quite beneficial to your metabolism and health. Presenting smaller amounts of nutrition continually, rather than massive quantities (and those all at once) appears to aid retention of nutrient uptake capacity. Drinking wine with meals is another French custom. The tradition is thought to aid in digestion. This may be important to nutrient uptake and longevity, as well. Living to the age of one hundred and twenty-two years, a French female holds the record for the longest life attained by a human.

Metabolism has long been known to be a factor in health and wellness. Within a species, a more efficient metabolism can lead to a much longer lifespan. Disorders of metabolism such as metabolic syndrome and diabetes are clearly shown to damage health and to shorten lifespan. Maintaining a healthy metabolism is key to health. Many and various studies affirm this. Trials with the fruit fly have illustrated that improving various aspects of intestine function can increase lifespan by as much as forty percent. Caloric restriction has been shown as being effective in prolonging lifespan within all animal species.

Caloric restriction was before thought to be effective in prolonging an individual's lifespan because it limits expenditures of energy and the resulting “wear and tear” on the body. The more important principle of this disclosure also applies. Caloric restriction works primarily by maximizing a species genetically predetermined capacity for nutrient uptake. Not overwhelming these uptake receptors and, in fact, not overwhelming the digestive system with excess food will improve health and increase survival.

The limiting aspect of lifespan expansion is the ability to uptake and to utilize nutrients. Elephants live a long time. Most could live even longer. However, many elephants ultimately succumb to starvation -simply due to their teeth wearing down.

In biological systems, prevention of wear preserves bodily resources. Avoiding wear conserves the resources and the nutritional reserve that would otherwise be needed to accomplish repairs. Conservation of resources, such as the bodily raw materials required for ordinary repair, is especially beneficial in older individuals. Recovery time after exercise, injury, or surgical procedures becomes much longer as individuals age. Simply put, older individuals who can stay healthy will live longer. Those who escape disease or serious injury will always outlive those survivors of major surgeries or those who have frequently become injured.

In the oldest of adults, extremely slow recovery from even the most minor bodily insult is common. The limiting factor in senior adult healing is not a simple slowdown in the mechanisms of cellular repair. The limiting factor is in the depletion of bodily reserves. The very old suffer from a generalized lower level of stored critical nutrients, the very nutrients essential for bodily repair.

There are two equally important and closely related factors at work within this disclosure. Slower healing is due from both a diminished capacity to absorb critical nutrients from the gut and to the body's need to cannibalize existing structures to obtain the needed repair materials. The inability to intake nutrients ultimately leads to a generalized shortage of raw materials within the body and the lack of easily available materials that can be used for repair.

In extreme aging the nutritional imbalance mimics the process of starvation. The aged body and a body dealing with starvation are alike in many important ways. In starvation, the body has an orderly process of shutting down non-vital functions to keep the individual alive until the current crisis is over. Neural hormones are the peptide structures that serve as messengers in the body. In the human body, nothing good will happen without something telling it to happen. The level of hormone decline is well documented in aging. The order of hormonal decline and loss of function is virtually indistinguishable in both systems, if not as well studied in starvation. Many processes of aging and of starvation are the same, if only slowed during the aging process.

Our bodies will not just “quit” when faced with diminished resources. A precise order of importance in resource utilization comes into play. In extreme aged individuals, the telomeres' of genes become shorter—not as a result of genetic damage. Telomeres' become foreshortened because in starvation mode the seemingly extra component is critically needed elsewhere. Changes in the genes are not a primary cause of aging. In this case, nutritional imbalance may be a cause of this change to the genes. In the interest of staying alive, the body will reabsorb all rarely used and less essential bodily components; including those immediately less important structures within a gene

### DESCRIPTION OF INFORMATION KNOWN

This disclosure ties together the inner workings of the corporate human body in support of an application for patent of a novel nutritional supplementation product. Nutritional supplements are not regulated as drugs in the United States of America. This disclosure goes beyond the statutory and regulatory requirements promulgated for these products. The intent of this additional material disclosure is to inspire further research into this and related fields of invention.

There are twenty amino acids comprising both bacterial and individual human cells composing the human body. The species-specific nutrient uptake limit is essentially a limit on the amount and kind of amino acids that may be absorbed by a multi-cellular organism during its lifetime. This lifetime limit will impact the single-cell bacteria that happen to be living in the gut of the larger organism, as well.

Serious scholarly work in basic science was halted by the many pressures on academia resulting from the outbreak of World War II. The field of amino acid science had been very fruitful prior to the war, with most of our knowledge, today, being based on work published during that period. The last truly novel work was published in 1942, but the included work was mostly done pre-war. After the war, new challenges emerged—serving to crystallize the prior work as orthodoxy. That earlier work, today, remains accepted and unchallenged.

According to this orthodoxy, the eight “essential” amino acids are: isoleucine, leucine, lysine, methionine, phenylalanine, threonine, tryptophan and valine. The “non-essential” amino acids are: proline, alanine, arginine, asparagin, aspartic acid, carnitine, citrulline, cysteine, cystine, gamma-amino butyric acid, glutamic acid, glutamine, glycine, omithine, serine, taurine, and tyrosine.

Other schemes, found in patent applications, further divide amino acids into: essential amino acids, e.g. isoleucine, leucine, lysine, methionine, phenylalanine, threonine, tryptophan, valine, or histidine, conditionally essential amino acids, e.g. tyrosine, cysteine, arginine, or glutamine, or non-essential amino acids, e.g. proline, glycine, alanine, serine, glutamic acid, aspartic acid, asparagines, taurine or camitine.

Other patent disclosures have amino acids broken down as small non-polar, hydrophobic amino acids that include proline, glycine, alanine, leucine, isoleucine, valine, and methionine. The large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine and the negatively charged (acidic) amino acids include aspartic acid and glutamic acid.

In spite of these noted differences, the general assumption in current research is that all amino acids are absorbed in the same way. In this disclosure, the term “amino acids” as used herein, unless otherwise stated, refers to amino acids in free form and/or in salt form chosen from one of the above classes. In this disclosure amino acids are accorded differing absorption characteristics in relation to these chemical and physical differences. This disclosure is of the differing absorption of amino acids over time

### Statement of Existing Problems Found in Prior Art

It has elsewhere been shown that metabolism and repair becomes less exact as an organism become older. The decline is real, if not the relationship to a cause. The observed decline may be due to a genetic change, a decline in nutrition or may be due to any of the multiple vectors of infectious disease. The decline noted may further be due to a combination of these factors. A physical decline eventually becomes noticeable with simple observation, even to the untrained eye. We may notice that we, physically, do not look as good nor do we move as well as we once did. We may notice that our bulk is increasing in some areas, or feel our metabolism is slowing. The first thought in these cases may be to eat less or to increase the amount exercise we are getting, as is advised in the prior art. Long term, neither course of action is certain to be therapeutic and, often, extremes of either may prove harmful.

The other area of decline, the decline in metabolic function, can be predicted, measured, and observed clinically. In a clinical setting, patients typically present first with hypertension, then metabolic syndrome, then “other” in a wide range of endocrine disorders. Hypertension causes “stroke” and metabolic syndrome can lead to diabetes. These disorders, however, can be treated clinically with success. The underlying cause of each of these syndromes is some “unknown” issue in the timely production of signaling hormones. This issue is a lack of the raw materials critical for peptide production.

Genetic changes that may, or may not, occur and what is broadly termed as “aging” are addressed elsewhere. This disclosure is of the mechanisms involved in the uptake of specific critical nutrients. Signaling peptides that control the feedback loops within the metabolism, as well as, the structural protein of rebuilding and repair have an important thing in common. In each process, the manufacture of protein is accomplished using specific component amino acids; each amino acid added to the next in a precise order. In both systems, proteins and peptides are precisely assembled combinations of among the twenty amino acids found within the body. The genes tightly control the entire process. If the body is lacking a specific amino acid, the organism cannot accomplish a specific metabolic process in a timely manner. If specific amino acids are in short supply the body cannot accomplish specific repair. Without the exact amino acid necessary for the next step, and without said amino acid being provided at the exact time it is needed, it can be said that “nothing good will happen in the body.”

The resultant change in bodily structure and function is not a symptom of “aging.” This is not Alzheimer's disease, nor is it arthritis. This is the effect of a lack of protein nutrition. Further, it begins much earlier than the development of the traditional “aging” symptoms, as described by others. Early on, the body is able to cover for a lack of absorption or for low absorption of certain amino acids by consuming plentiful reserves. The body may, at first, simply be obtaining needed amino acids from areas where their loss is less visible, such as from the outside covering of blood vessels. The consumption of critical reserves for standard bodily maintenance and the continuation of normal function are the twin forces that combine to compromise health and wellness and to foreshorten longevity.

The prior art descriptive of decline as we grow older universally revolves around genetic change. This is because our genes are expected to maintain a tight control over the manufacture of protein within the body. One current and respected theory involves “tired genes.” It notes that some genes just wear out. Another theory places a limit on the number of times that a gene may be successfully copied. Other theories cite a culmination of genetic errors as the causes of the multitude of symptoms associated with that which is termed to be “normal aging.” A multiplicity of theories claim to account for the various symptoms associated with aging but none, thus far, account for the entire phenomenon.

No prior art on “Aging” takes into account, or can explain all of the changes that seem to be occurring, as we grow older. Interestingly, the current most popular theory on the causes of the physical and functional decline associated with growing older involves changes to an area of the gene that does not encode anything. Clearly, the generic component of the process deserves to be revisited.

In one disclosure, many and diverse symptoms are lumped together under the term “Ageing.” The same experiments are repeated again and again and the decline of genes with every replication is calculated with precision. The design of these experiments do not account for the “real world” variability of aging symptoms. What has actually been observed is quite different from that which is claimed.

There is another historical perspective. The post World War progress made in penicillin and other antibiotics limited our concerns about bacteria. Other than diarrhea, a common side effect of antibiotics on our own beneficial bacteria, bacteria have become not of concern to us. Depending on how it is counted, there are ten to one-hundred times the number of bacterial cells living in or on our bodies, as there are cells in our own body. Bacteria living in the gut of an animal will play an important part in the nutrition of that animal. Humans are no exception to this natural law.

Young animals with “knock-out” genes that cause them to bear a physical resemblance to aging adults are not actual aged individuals. Their intestines are young intestines and these intestines are filled with active, young bacteria. These young animals may very easily benefit from the addition of low levels of nutrients that are difficult for older intestines populated with old and damaged bacteria to absorb.

Genetic change, as it relates to the longevity claims in prior art, needs to be addressed. The basis of this disclosure is contemporaneous genetic change is largely not responsible for the disease and the physical decline associated with growing older. As we grow older, many changes to processes under genetic control do occur. This is, again, why the genes are so often faulted. Genes, by themselves, are unable to cause every change that may or can occur. Much of the decline observed, both physical and metabolic, is due to a lack of absorption of specific nutrition. This lack of uptake leads to a depletion of bodily reserves and to the likely repeat of the observed error. The observation of this phenomenon is further compounded by the individual bodily responses to the lack of utility from the unmade protein. This accounts for the high variability of the signs and symptoms of disease observed within individuals as they begin to grow older.

Many of the observable and reported genetic transcription errors and omissions are due to the shortage of a specific amino acid needed to fully complete that specific process. This is why the progression of symptoms accompanying the attainment of a great age is more akin to the progression of change seen during the process of starvation. A slow starvation by the mechanism of a lack of intake and the depletion of bodily reserves is described in this disclosure.

1. I claim oral nutritional supplement products for the purpose of improvement of nutritional reserves containing L-Proline as the sole active ingredient.

2. The method of claim 1 where the content of L-Proline is greater than 5 percent by weight.

3. The method of claim 1 where the content of L-Proline is greater than 6 percent by weight.

4. I claim oral nutritional supplement products for the purposes of improvement of nutritional reserves that are alcoholic or hydro-alcoholic preparations containing L-Proline.

5. The method of claim 4 where the L-Proline content is greater than 5% by weight or w/v

6. The method of claim 4 where the L-Proline content is greater than 6% by volume or w/v

7. The method of claim 4 when used as adjunct therapy to rid the body of herpes infections.

8. The method of claim 4 when used as therapy to reduce the metabolic symptoms of aging.

9. The method of claim 4 when used as therapy in wasting diseases.

10. The method of claim 4 when used to prepare the female body for pregnancy or menses.

11. The method of claim 4 when used as an adjunct to reverse metabolic decline.

12. The method of claim 4 when used as therapy for collagen diseases.

13. The method of claim 4 when used as in combination with other amino acids.

14. The method of claim 4 when used as a beverage.

15. I claim intravenous, parental and enteral amino acid preparations that include a preponderance of the amino acid L-proline as an active ingredient

16. The method of claim 15 excepting and specifically excluding full complement amino acid products or intravenous solutions containing proline in amounts less than one hundred percent greater than the percentage concentration of proline in current and commercially available full complement amino acid intravenous solutions.

17. The method of claim 4 when used to as a vehicle for other drugs.

18. The method of claim 4 when used to promote longevity.

19. The method of claim 4 when used to improve health and wellness and/or to strengthen or improve metabolism.

20. The method of claim 4 when used to improve the appearance of the skin
